(19) United States
(12) Patent Application Publication
(10) Pub. No.: US 2025/0387532 A1
(43) Pub. Date: Dec. 25, 2025

(54) IMPLANTABLE ABSORBABLE TISSUE ADHESIVES ON BIOMATERIALS FOR LONG-TERM ADHESION TO BIOLOGICAL TISSUES

(71) Applicant: W. L. Gore & Associates, Inc., Newark, DE (US)

(72) Inventors: Kyle R. Buckley, Flagstaff, AZ (US); Amit K. Jha, Bellemont, AZ (US); Peter D. Traylor, Flagstaff, AZ (US); Clayton J. Underwood, Flagstaff, AZ (US); Brannon R. McCullough, Flagstaff, AZ (US)

(21) Appl. No.: 19/243,107

(22) Filed: Jun. 19, 2025

Related U.S. Application Data
(60) Provisional application No. 63/661,787, filed on Jun. 19, 2024.
(60) Provisional application No. 63/825,863, filed on Jun. 18, 2025.

(51) Int. Cl. A61L 27/34; A61L 27/56; A61L 27/58

(52) U.S. Cl. CPC A61L 27/34; A61L 27/56; A61L 27/58

(57) ABSTRACT

The present disclosure relates generally to compositions, systems, and methods for vascular closure, such as upon applying an implantable article to biological tissue and thereby adhering said article to the tissue to which it has been applied. More specifically, the disclosure relates to compositions, systems, and methods that involve articles modified with crosslinkable compositions, the compositions facilitating crosslinking between the article and biological tissue at physiological pH and temperature.

### CROSS-REFERENCE TO RELATED APPLICATION

This application claims the benefit of Provisional Application No. 63/661,787, filed Jun. 19, 2024, and also claims the benefit of Provisional Application No. 63/825,863, filed Jun. 18, 2025, which are incorporated herein by reference in their entireties for all purposes.

### FIELD

The present disclosure relates generally to adhesive implantable compositions, systems, and methods relating to vascular closure. Specifically, the disclosure relates to compositions, systems, and methods relating to closure of and/or adhesion to biological tissue.

### BACKGROUND

Vascular closure is a vital component of a vascular surgery, which involves, e.g., medical device implantation, aneurysm repair, or providing percutaneous circulatory support. Migration of implantable medical devices from the site of in vivo implantation can disrupt device function, potentially resulting in arrhythmias, syncope or discomfort for the patient, and severe bioresponses. Thus, long term fixation of medical devices after the implantation is required.

Suturing is traditional solution to fix the migration of medical devices at the implantation site, however, invasive suturing to prevent migration of medical devices slows down subsequent tissue healing and may inhibit the integration of device with tissue due to infection, inflammation, and chronic bioresponses. Furthermore, suturing is a focal fixation technique that can only be applied to specific regions on an implant. There is a need for adhesive compositions, systems, and methods that are capable of adhering the entire implant surface to the surrounding tissue. Additionally, there is a need for adhesive compositions, systems, and methods that are capable of providing an effective seal in the vasculature to prevent possible leakage or infection.

### SUMMARY

Implantable materials are provided to have a surface and a coating on at least a portion of the surface, where the surface can be an inner or an outer surface. In some examples, the coating adheres, such as crosslinks, to a biological material in the presence of a crosslinking agent. In additional examples, the coating is capable of adhering the implantable material directly to a biological tissue, e.g., via crosslinking upon contact with blood or saline. As discussed herein, the materials, systems, methods, and kits involving such implantable materials may be used in various applications, including but not limited to surgical applications. The disclosed materials, systems, methods, and kits may be used in conjunction with pusher sleeves/sheaths, introducers, guidewires, and/or any other suitable devices for introducing or implanting medical devices and apparatuses into and out of the body during surgical procedures.

According to one example (“Example 1”), implantable article including a bioabsorbable material is provided. The implantable article including a bioabsorbable material, the bioabsorbable material including a surface and a coating on at least a portion of the surface, where the coating includes a crosslinkable composition, the crosslinkable composition including an amine functional polymer immobilized to the surface by crosslinking with a compound including an aldehyde group, and wherein the surface can be an inner or outer surface.

According to another example (“Example 2”), further to Example 1, the bioabsorbable material includes poly (α-hydroxy esters), poly(lactic-co-glycolic acid) (PLGA), poly(glycolic acid) (PGA), poly(lactic acid) (PLA), trimethylene carbonate (TMC), or a combination thereof.

According to another example (“Example 3”), further to any of the preceding Examples, the amine functional polymer includes polyethylenimine (PEI), pol (allylamine), poly(L-lysine), chitosan, poly(4-aminostyrene), poly(N-methylvinylamine), Oleylamine, lenoeylamine, dodecylamine, tris[(3 msalicylideneimino) ethyl]amine, polyvinylamine or a combination thereof; or where the amine functional polymer includes an amine modified polymer selected from a poly(ethylene glycol)-amine, Poly(N-isopropylacrylamide) amine terminated, poly(ethylene glycol) bis(3-aminopropyl) terminated, amine functionalized hyaluronic acid, gellan gum, or cellulose.

According to another example (“Example 4”), further to any of the preceding Examples, the crosslinkable composition further includes a crosslinking intermediate, the crosslinking intermediate including an amine group, a succinimide group, a carbonyl group, an aldehyde group, an ester group, an acrylate group, a methacrylate group, a hydroxyl group, a carboxyl group, an isocyanate group, a sulfonyl group, or a thiol group.

According to another example (“Example 5”), further to any of the preceding Examples, the implantable article has a three-dimensional structure, the three dimensional structure including a planar structure, an ellipsoid, a hemi-ellipsoid, a partial ellipsoid, a cone, a partial dome, a tubular construct, a sphere, a hemisphere, a partial sphere, a spheroid, a hemispheroid, or a partial spheroid.

According to another example (“Example 6”), further to any of the preceding Examples, the bioabsorbable material includes a porosity of greater than 70%, greater than 80%, or greater than 90%.

According to another example (“Example 7”), further to any of the preceding Examples, the bioabsorbable material includes poly(lactic acid) (PLA), poly(glycolic acid) (PGA), poly(lactic-co-glycolic acid) (PLGA), polydioxanone (PDO), polycaprolactone (PCL), poly(α-hydroxy esters), trimethylene carbonate (TMC), poly(L-lactide-co-ϵ-caprolactone) (PLCL), poly(D,L-lactic acid) (PDLLA), poly(L-lactic acid) (PLLA), poly(D-lactic acid) (PDLA), poly(glycolide-co-trimethylene carbonate) (PGTMC), polyethylene terephthalate (PET), polyhydroxyalkanoates (PHAs), hyaluronic acid, collagen, gelatin, cellulose, polyethylene, polypropylene, poly(propylene sulfide), poly(thioether), polyether, poly(thioketal), poly (L-proline), poly (L-methionine), or a combination thereof, and the amine functional polymer includes polyethylenimine covalently immobilized to the outer surface by crosslinking with glutaraldehyde.

According to another example (“Example 8”), an implantable article including a non-absorbable material is provided. The implantable article including a non-absorbable material has a surface and a coating on at least a portion of the surface, the coating includes a crosslinkable composition, the crosslinkable composition including an amine functional polymer immobilized to the surface by crosslinking with a compound including an aldehyde group, and wherein the surface can be an inner or outer surface.

According to another example (“Example 9”), and further to Example 8, the non-absorbable material includes polyurethane (PU), polyamide (PA), polytetrafluoroethylene (PTFE), polyetheretherketone (PEEK), polyaryletherketone (PAEK), polyetherimide (PEI), polyethersulfone (PES), polyetherketoneketone (PEKK), an expanded polymer thereof, or a combination thereof.

According to another example (“Example 10”), and further to Example 8 or 9, the amine functional polymer includes polyethylenimine, poly(allylamine), poly(L-lysine), chitosan, poly(4-aminostyrene), poly(N-methylvinylamine), Oleylamine, lenoeylamine, dodecylamine, tris[(3 msalicylideneimino) ethyl] amine, polyvinylamine or a combination thereof; or the amine functional polymer includes an amine modified polymer selected from a poly(ethylene glycol)-amine, Poly(N-isopropylacrylamide) amine terminated, poly(ethylene glycol) bis(3-aminopropyl) terminated, amine functionalized hyaluronic acid, gellan gum, or cellulose.

According to another example (“Example 11”), and further to Examples 8-10, the crosslinkable composition further includes a crosslinking intermediate, the crosslinking intermediate including an amine group, a succinimide group, a carbonyl group, an aldehyde group, an ester group, an acrylate group, a hydroxyl group, a carboxyl group, a sulfonyl group, or a thiol group.

According to another example (“Example 12”), and further to Examples 8-11, the implantable article has a three-dimensional structure, the three dimensional structure including a planar structure, an ellipsoid, a hemi-ellipsoid, a partial ellipsoid, a cone, a partial dome, a tubular construct, a sphere, a hemisphere, a partial sphere, a spheroid, a hemispheroid, or a partial spheroid.

According to another example (“Example 13”), and further to Examples 8-12, the implantable article further includes a coating shield configured to delay or prevent for about 1 to about 15 mins premature reactivity with a bodily fluid including a protein.

According to another example (“Example 14”), and further to Examples 8-13, the protein is a blood protein.

According to another example (“Example 15”), and further to Examples 8-14, the implantable article has a coating shield, the coating shield including a chemical shield, a mechanical shield, or a combination thereof.

According to another example (“Example 16”), and further to Examples 8-15, the chemical shield includes a second composition including a negatively charged polymer functionalized with crosslinkable groups.

According to another example (“Example 17”), and further to Examples 8-16, the second composition is on at least a portion of the coating that is on the outer surface of the bioabsorbable material.

According to another example (“Example 18”), and further to Examples 8-17, the negatively charged polymer includes carboxyl groups, hydroxyl groups, sulfonate groups, or a combination thereof, and wherein the crosslinkable groups include amine reactive functional groups including N-hydroxy succinimide groups, aldehyde groups, isocyanate groups, or a combination thereof.

According to another example (“Example 19”), and further to Examples 8-18, the implantable article further including hydrophobic molecules or hydrophobic polymers.

According to another example (“Example 20”), and further to Examples 8-19, the hydrophobic molecules or hydrophobic polymers are applied to the second composition that is on the outer surface of the bioabsorbable material or co-formulated with the second composition.

According to another example (“Example 21”), and further to Examples 8-20, the mechanical shield includes a knitted zipper cover, a delivery sleeve, or a delivery sheath cover.

According to another example (“Example 22”), and further to Examples 8-21, the knitted zipper cover includes expanded polytetrafluoroethylene (ePTFE), expanded polyethylene (ePE), polyether ether ketone (PEEK), ultra-high molecular weight polyethylene (UHMW PE), polysulfone (PSU), poly(methyl methacrylate) (PMMA), polypropylene (PP), polyethylene terephthalate (PET), polylactic acid (PLA), polycaprolactone (PCL), polyurethane (PU), silicone, nylon, polyvinylidene fluoride, perfluoroalkoxy alkane, fluorinated ethylene propylene, or combinations thereof, and the knitted zipper cover has a knit density of about 20 to about 70 stitches per inch.

According to another example (“Example 23”), a tissue adhesion system is provided. The tissue adhesion system includes an implantable article including a surface including a crosslinkable composition, a multi-branched or multivalent crosslinker composition, and a biological tissue, where the implantable article is operable to be placed into contact with the biological tissue, and wherein the multi-branched crosslinker composition facilitates crosslinking between the crosslinkable composition and the biological tissue, thereby operable to adhere the implantable article to the biological tissue, and wherein the surface can be an inner or outer surface.

According to another example (“Example 24”) and further to Example 23, the implantable article includes a bioabsorbable material, a non-absorbable material, or a combination thereof.

According to another example (“Example 25”) and further to Example 23, the bioabsorbable material includes poly(lactic acid) (PLA), poly(glycolic acid) (PGA), poly(lactic-co-glycolic acid) (PLGA), polydioxanone (PDO), polycaprolactone (PCL), poly(α-hydroxy esters), trimethylene carbonate (TMC), poly(L-lactide-co-ϵ-caprolactone) (PLCL), poly(D,L-lactic acid) (PDLLA), poly(L-lactic acid) (PLLA), poly(D-lactic acid) (PDLA), poly(glycolide-co-trimethylene carbonate) (PGTMC), Polyethylene terephthalate (PET), polyhydroxyalkanoates (PHAs), hyaluronic acid, collagen, gelatin, cellulose, polyethylene, polypropylene, poly(propylene sulfide), poly(thioether), polyether, poly(thioketal), poly(L-proline), poly(L-methionine), or a combination thereof.

According to another example (“Example 26”) and further to Example 24, the non-absorbable material includes, polyurethane (PU), polyamide (PA), polytetrafluoroethylene (PTFE), polyetheretherketone (PEEK), polyaryletherketone (PAEK), polyetherimide (PEI), polyethersulfone (PES), polyetherketoneketone (PEKK), or a combination thereof.

According to another example (“Example 27”) and further to Example 23-26, where the crosslinkable composition is associated with the outer surface of the implantable article by covalent immobilization, electrostatic adsorption, or hydrogen bonding.

According to another example (“Example 28”) and further to Example 23-27, the crosslinkable composition includes an amine group, a succinimide group, a carbonyl group, an aldehyde group, an ester group, an acrylate group, a hydroxyl group, a carboxyl group, a sulfonyl group, a thiol group, or a combination thereof.

According to another example (“Example 29”) and further to Example 28, the crosslinkable composition includes the amine functional group, the carbonyl group, the aldehyde group, the ester group, the acrylate group, the hydroxyl group, the carboxyl group, the sulfonyl group, or the thiol group at a density of at least 1 μmol/cm², 10 μmol/cm², 100 μmol/cm², 250 μmol/cm², 500 μmol/cm², 750 μmol/cm², or 1000 μmol/cm².

According to another example (“Example 30”) and further to Examples 23-29, the crosslinkable composition includes polyethyleneimine, polyethylenimine, poly(allylamine), poly(L-lysine), chitosan, dextran, vinyl sulfone dextran, chitosan, gelatin, hyaluronic acid, or a combination thereof.

According to another example (“Example 31”) and further to Examples 23-30, the biological tissue includes an amine group, a carboxylic acid group, a hydroxyl group, a thiol group, or a combination thereof.

According to another example (“Example 32”) and further to Examples 23-31, the multi-branched crosslinker composition includes an amine-functionalized polymer or copolymer, a reactive PEG derivative, a reactive natural product or derivative, or a combination thereof.

According to another example (“Example 33”) and further to Example 32, the reactive PEG derivative includes poly(ethylene glycol) N-hydroxysuccinimide ester (PEG-NHS), multi-arm PEG-NHS, poly(ethylene glycol) aldehyde (PEG-CHO), multi-arm PEG-CHO, or PEG-maleimide.

According to another example (“Example 34”) and further to Example 32, the reactive natural product or derivative includes a quinone, a flavonoid, a coumarin, or a lignin.

According to another example (“Example 35”) and further to Example 34, the reactive natural product or derivative includes genipin, tannic acid, or glyoxal.

According to another example (“Example 36”) and further to Examples 23-35, the multi-branched crosslinker composition includes a solvent, a buffer, or a pharmaceutically acceptable excipient.

According to another example (“Example 37”), and further to Examples 23-36, the implantable article further includes a coating shield configured to delay or prevent for about 1 to about 15 mins premature reactivity with a bodily fluid including a protein.

According to another example (“Example 38”), and further to Examples 23-37, the protein is a blood protein.

According to another example (“Example 39”), and further to Examples 23-38, the implantable article has a coating shield, the coating shield including a chemical shield, a mechanical shield, or a combination thereof.

According to another example (“Example 40”), and further to Examples 23-39, the chemical shield includes a second composition including a negatively charged polymer functionalized with crosslinkable groups.

According to another example (“Example 41”), and further to Examples 23-40, the second composition is on at least a portion of the coating that is on the outer surface of the bioabsorbable material.

According to another example (“Example 42”), and further to Examples 23-41, the negatively charged polymer includes carboxyl groups, hydroxyl groups, sulfonate groups, or a combination thereof, and wherein the crosslinkable groups include amine reactive functional groups such as N-hydroxy succinimide groups, aldehyde groups, isocyanate groups, or a combination thereof.

According to another example (“Example 43”), and further to Examples 23-42, the implantable article further including hydrophobic molecules or hydrophobic polymers.

According to another example (“Example 44”), and further to Examples 23-43, the hydrophobic molecules or hydrophobic polymers are applied to the second composition that is on the outer surface of the bioabsorbable material or co-formulated with the second composition.

According to another example (“Example 45”), and further to Examples 23-44, the mechanical shield includes a knitted zipper cover, a delivery sleeve, or a delivery sheath cover.

According to another example (“Example 46”), and further to Examples 23-45, the knitted zipper cover includes expanded polytetrafluoroethylene (ePTFE), expanded polyethylene (ePE), polyether ether ketone (PEEK), ultra-high molecular weight polyethylene (UHMW PE), polysulfone (PSU), poly(methyl methacrylate) (PMMA), polypropylene (PP), polyethylene terephthalate (PET), polylactic acid (PLA), polycaprolactone (PCL), polyurethane (PU), silicone, nylon, polyvinylidene fluoride, perfluoroalkoxy alkane, fluorinated ethylene propylene, or combinations thereof, and the knitted zipper cover has a knit density of about 20 to about 70 stitches per inch.

According to another example (“Example 47”) a kit is provided. The kit includes the implantable article according to Examples 1-22 and a multi-branched crosslinker composition.

According to another example (“Example 48”), a method for rapidly adhering the implantable article of any one of Examples 1-22 to an adjacent tissue during a surgical procedure is provided. The method includes surgically implanting the implantable article by positioning the implantable article to an adjacent tissue in a subject, applying a multi-branched or multivalent crosslinker composition to at least a portion of the coating on at least a portion of the surface of the implantable article, thereby adhering the implantable article to the adjacent tissue, and wherein the surface can be an inner or outer surface.

According to another example (“Example 49”) and further to Example 48, the surgical procedure includes implantation of suture-less hernia mesh, implantation of a fistula plug, filling a diabetic foot ulcer wound, repairing a thoracic aortic aneurysm with no landing, repair of av fistula, defect closure in aorta, sealing of air-leaking lung lobe, sealing of air-leaking trachea, adhesion of drug patch/sensor to an organ, anastomosis of small intestine, sealing of fluid-leaking stomach, sealing and landing devices in large blood vessels, septal defect closure, aortic dissection repair, or fixation of a tissue reinforcement graft.

According to another example (“Example50”) and further to Examples 48 or 49, the adhesion between the implantable bioabsorbable material or the implantable article and the adjacent tissue has an adhesion strength of at least 0.4 mPA, 0.5 mPA, 0.6 mPA, 0.7 mPA, or 0.8 mPA.

According to another example (“Example 51”) and further to Examples 48-50, the subject is a mammal.

According to another example (“Example 52”) and further to Example 51, the mammal is human.

According to another example (“Example 53”) a method for preparing an implantable article according to any of the preceding Examples is provided. The method includes providing an implantable article, optionally including a bioabsorbable material or a non-absorbable material, having a surface, and applying a coating on at least a portion of the surface, where the coating includes an amine functional polymer immobilized to the surface, the amine functional polymer immobilized to the surface by crosslinking with a compound including an aldehyde functional group, wherein the surface can be an inner or outer surface.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows a diagram of an implantable article 100 having a body 102, an outer surface 104, and a coating 106 containing a crosslinkable composition as described herein.

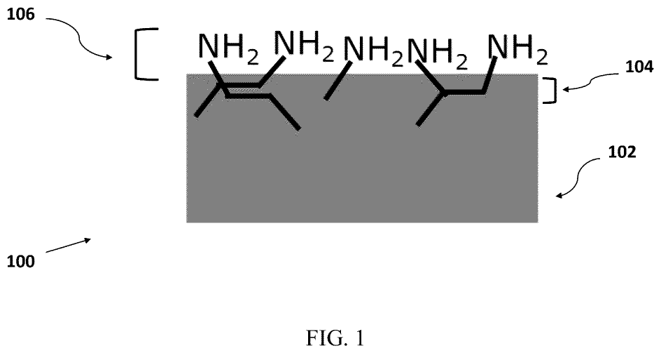

FIG. 2 shows the adhesive strength of an embodiment of an implantable bioabsorbable material to aorta tissue.

FIG. 3 shows the adhesive strength of implantable bioabsorbable materials to aorta tissue, where the implantable bioabsorbable material is coated with a first layer of PEI and a second layer of PEI, where the first and second coatings contain PEI at the same or distinct molecular weights.

FIG. 4 shows the adhesive strength of an embodiment of an implantable bioabsorbable material to aorta tissue following application of multi-branched crosslinker composition containing multi-arm PEG-NHS (4 and 8 arms) at various PEG molecular weights, including 5 kDa, 10 kDa, and 20 kDa.

FIG. 5 shows the adhesive strength of an embodiment of an implantable bioabsorbable material to aorta tissue, where the concentration of 4-arm 10 kDa and 20 kDa PEG MW PEG-NHS is varied from 20 wt % and 40 wt %.

FIG. 6 shows the adhesive strength of an embodiment of an implantable bioabsorbable material to aorta tissue, where the concentration of NHS-functionalized PAA was 2.5 wt %, 5.0 wt%, 10.0 wt %, and 15.0 wt %.

FIG. 7 shows the adhesive strength of an embodiment of an implantable article including bioabsorbable or nonabsorbable material to aorta tissue, where the coating including a crosslinkable composition was applied to ePTFE, ePE, PET, or NexED BioA.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

As used herein a “bioabsorbable material” or “bioabsorbable article” refers to a material or device that is capable of being broken down and absorbed by the body over time through natural biological processes. The terms may be used interchangeably with “biodegradable,” “bioabsorbable,” “bioresorbable,” “resorbable,” and the like, which describe how the bioabsorbable material or article can be decomposed by hydrolysis, reactive oxygen species (ROS) such as hydroxyl radicals (·OH), superoxide (O2-), hydrogen peroxide (H2O2), and hypochlorite (ClO-) or biological agents, such as enzymes and microorganisms, and bioresorbable, indicating that the material or article be gradually dissolved and eliminated from the body of a mammal by any of a variety of mechanisms, e.g., phagocytosis.

As used herein a “non-absorbable material” or “non-absorbable article” generally refers to a substance that is resistant to degradation under physiological conditions within the body, i.e., the non-absorbable material is one that is neither degraded nor absorbed by the body over time. It may be inert, biocompatible, and remain intact indefinitely, such as after being implanted or introduced into the body of a mammal.

As used herein “non-woven” generally refers to a type of fabric or substrate made directly from fibers or filaments or from a web of fibers without the preliminary filament preparation needed for weaving, knitting, or braiding.

As used herein “inelastic” generally refers to a material property in which the material substantially resists elongation or lengthening.

As used herein “tissue”, “cell tissue” or “biological tissue” generally refers to associations of cells which consist of cells of the same form and function, such as surface tissue (skin), epithelial tissue, myocardial, connective or stromal tissue, muscles, nerves and cartilage. This also includes, among other systems, all organs made up of associations of cells, such as the liver, kidneys, lungs, heart, etc.

As used herein “premature reactivity with a bodily fluid” generally refers to the process of forming crosslinking bonds between the amine reactive functional groups on the surface of the implantable article and the free amine groups in a bodily fluid including proteins.

As used herein “coating shield” generally refers to a chemical or mechanical coating that is used to prevent or delay the coating including a crosslinkable composition of the implantable article from coming into contact with a bodily fluid including a protein and prematurely reacting with the protein before reaching the intended adhesion location.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

In various examples, adhesive compositions, systems, methods, and kits according to this patent specification may be used in association with various medical procedures, including in association with endoluminal (e.g., endovascular procedures) that require access to one or more body lumens through an access site. The puncture, or wound, to the skin, tissue, and lumen proximate the access site may be treated through use of such adhesive compositions, systems, and methods. For example, the adhesive compositions, systems, and methods may assist in short term wound closure and/or longer term healing.

In some embodiments, the system may be a tissue adhesion system including an implantable article. The implantable article has an outer surface that has a coating, where the coating includes a crosslinkable composition. In some embodiments, the implantable article has an inner surface that has a coating, where the coating includes a crosslinkable composition. The crosslinkable composition is capable of crosslinking to a crosslinkable moiety which is supplied by a biological tissue or fluid, such as a biological interface of proteins including amino acids having reactive moieties capable of crosslinking. In some examples, crosslinking between the crosslinkable composition and the biological tissue adheres the implantable article to the biological tissue.

The inner or outer surface of the implantable article can be coated with a single layer of coating or several layers of coating including a crosslinkable composition. The crosslinkable composition includes at least one crosslinkable moiety, such as a first crosslinkable moiety, a second crosslinkable moiety, or a third crosslinkable moiety. The crosslinkable composition can be associated with the inner or outer surface of the implantable article. Such association with the inner or outer surface of the implantable article may be achieved by covalent immobilization, electrostatic adsorption, or hydrogen bonding.

Additionally, or alternatively, the implantable article may be a blood-activated adhesive, or a saline-activated adhesive, the adhesiveness of which is activated in response to being exposed to an environment that is filled with blood or saline solution. In such cases, the implantable article is activated in a blood-filled environment. The implantable article may be applied between the body of a closure device and the surrounding tissue wall of the vasculature (artery) such that exposing the implantable article to the blood within the vasculature is sufficient to activate the adhesive properties of the inner or outer surface of the implantable article.

As described, an implantable article can have several layers of coating including a crosslinkable composition. For example, the coating can include at least a first layer, a second layer, a third layer, a fourth layer, etc. of such a coating. In order to tune adhesion strength and speed of adhesion, each layer can include similar or distinct components.

The crosslinkable composition may include a polymer having a reactive functional group, including but not limited to: an amine group, a carbonyl group, an aldehyde group, an ester group, an acrylate group, a hydroxyl group, a carboxyl group, a sulfonyl group, a thiol group, or a combination thereof. In certain non-limiting examples, the polymer can be polyethyleneimine, polyethylenimine (PEI), poly(allylamine), poly(L-lysine), or combinations thereof. Herein a “crosslinking composition” may be used interchangeably with the term “crosslinkable composition.”

A polymer present in the crosslinkable composition can have a molecular weight that is at least 1000 Da, 2000 Da, 4000 Da, 5000 Da, 8000 Da, 10,000 Da, 15,000 Da, 20,000 Da, 25,000 Da, or 50,000 Da. The polymer can have a molecular weight ranging from 1000-10,000 Da, 2000-25,000 Da, 4000-50,000 Da, 2000-10,000Da, 1500-5000 Da, or 5000-25,000 Da.

Functional group density may be expressed in units of micromoles per square centimeter. The crosslinking composition can include a polymer having a functional group density, such as any of a carbonyl group, an aldehyde group, an ester group, an acrylate group, a hydroxyl group, a carboxyl group, a sulfonyl group, or a thiol group, of at least 1 μmol/cm², 10 μmol/cm², 100 μmol/cm², 250 μmol/cm², 500 μmol/cm², 750 μmol/cm², or 1000 μmol/cm². For example, the density of a functional group can range from 1-1000 μmol/cm², 10-950 μmol/cm², 50-900 μmol/cm², 100-850 μmol/cm², 150-800 μmol/cm², 200-750 μmol/cm², 250-700 μmol/cm², 300-650 μmol/cm², 350-600 μmol/cm², or 400-550 μmol/cm².

Alternatively, the crosslinking composition can include a polymer having a functional group density of at least 10 μmol/cm³, 100 μmol/cm³, 1,000 μmol/cm³, or 10,000 μmol/cm³. For example, the crosslinking composition can include a polymer having a functional group density ranging from 10-15,000 μmol/cm³, 10-10,000 μmol/cm³, 100-10,000 μmol/cm³, 1000-15,000 μmol/cm³, 5000-15,000 μmol/cm³, or 10,000-15,000 μmol/cm³.

The physical characteristics of each successive layer of coating containing the crosslinking composition can be adjusted. For example, the concentration of polymers and the physical characteristics thereof can be altered depending on the layer. In one illustrative example, an initial layer of crosslinking composition can include PEI having a molecular weight of 20 kDa, the second layer of coating can include a crosslinking composition with PEI having a molecular weight of 25 kDa, and the third layer of coating can include a crosslinking composition with PEI having a molecular weight of 50-100 kDa. In other examples, each layer can include distinct polymers of varied or similar molecular weight and concentration.

Certain variables may be altered to achieve improved adhesion of the implantable article, such as adhesion strength, speed of adhesion, and the like. Such variables include altering the relative ratio of unprotonated and protonated functional groups available for crosslinking. Unprotonated amines are preferred for crosslinking reactions because the nucleophilic lone pair of electrons on the nitrogen atom is available to react with electrophilic crosslinking reagents. In contrast, protonated amines lack this reactive lone pair. The unprotonated functional groups and protonated functional groups involved in covalent permanent bonding impart temporary electrostatic bonding and so it is advantageous to increase the amount of unprotonated amines in a coating, such as an outer layer of a coating, on an implantable article. Increasing the ratio of unprotonated to protonated amines can be achieved, for example, by treating the outer layer of an implantable article having PEI coating with borax or boric acid/sodium monophosphate to convert protonated amines into unprotonated amines.

The ratio of unprotonated to protonated amines in polymers, like PEI, can be expressed using the protonation fraction or degree of protonation (a). The protonation fraction (a) represents the fraction or percentage of amine groups that are protonated (positively charged) in the polymer at a given pH or solution condition. An a value of 0 indicates that all amine groups are unprotonated (neutral), and an a value of 1 (or 100%) indicates that all amine groups are fully protonated. Accordingly, an a value between 0 and 1 is indicative of a partially protonated state having a mixture of protonated and unprotonated amines. For example, at physiological pH (7.4), the outer coating of an implantable article may be characterized as having an a value of less than 1, 0.9, 0.8, 0.7, 0.6, or 0.5, and may range from 0-0.9, 0.1-0.8, 0.1-0.5, 0.2-0.7, 0.2-0.4, 0.3-0.6, or 0.4-0.5.

Adhesion, as facilitated by crosslinking, can be activated by the temperature and pH of the reaction conditions. Certain crosslinking reactions involve the formation of reactive intermediates that can be activated by the physiological temperature of blood (around 37° C.). In one illustrative example, N-Hydroxysuccinimide (NHS) esters, crosslinking reagents which react with primary amines, are more reactive at higher temperatures, facilitating crosslinking at body temperature, about 37° C. The pH of blood (around 7.4) can also activate certain crosslinking intermediates. The functionalized monomer is “activated” by the temperature and mildly basic pH of blood, thereby enabling crosslinking reactions to occur within biological system. The crosslinking reaction in turn results in adherence of the implantable article directly to the biological tissue it is applied to.

The crosslinking composition can include a crosslinking intermediate that is activated under certain conditions, such as the physiological conditions characterized by exposure to body temperature and blood pH. In embodiments, the crosslinking intermediate may be activated at pH of greater than 7 or less than 12. For example, the crosslinking intermediate may be activated at a pH ranging from 7-12, 7-10, 7-9, 8-12, 8-10, or 10-12.

The crosslinking intermediate can be associated with the polymer in the crosslinkable composition. For example, the crosslinking intermediate can be crosslinked to or otherwise associated with the polymer such that it functionalizes the polymer.

The crosslinking composition may include a crosslinking intermediate that includes an amine group, a carbonyl group, an aldehyde group, an ester group, an acrylate group, a methacrylate group, a hydroxyl group, a carboxyl group, an isocyanate group, a sulfonyl group, or a thiol group. In one illustrative example, NHS functionalized polyethylene glycol (PEG) can be crosslinked to unprotonated amine functional groups on immobilized PEI on the surface of an implantable article as described herein, and the NHS-PEG can then adhere to a biological tissue, such as in the presence of a biological fluid, like blood. Exemplary crosslinking intermediates include dextran and derivatives thereof, chitosan, gelatin, and hyaluronic acid. The crosslinkable composition can include bi-functionalized or muti-functionalized PEG, NHS derivatized PEG (PEG-NHS), PEG aldehyde (PEG-CHO), and PEG and other polymers derivatives with other polymerizable groups, such as isocyanate, acrylates and acrylamides.

In some examples, the crosslinking intermediate is omitted from an initial layer of coating but is included in each successive layer of coating. In other examples, the crosslinking intermediate is included in each layer of coating. In other examples, the crosslinking intermediate is included only in the outermost layer of coating, such as the last applied layer of coating including a crosslinking composition as described. Pore size and thickness of substrate, molecular weight and polymer branching influence coating choice to ensure accessible functional groups for bonding with tissue.

A layer of the crosslinkable composition coating the implantable article may include an amine functional polymer immobilized to the inner or outer surface, the amine functional polymer immobilized to the inner or outer surface by crosslinking with a compound including an aldehyde functional group. The amine functional polymer may include polyethylenimine, poly(allylamine), poly(L-lysine), chitosan, poly(4-aminostyrene), poly(N-methylvinylamine), Oleylamine, lenoeylamine, dodecylamine, tris[(3 msalicylideneimino)ethyl] amine, polyvinylamine or a combination thereof, or include an amine modified polymer selected from a poly(ethylene glycol)-amine, Poly(N-isopropylacrylamide) amine terminated, poly(ethylene glycol) bis(3-aminopropyl) terminated, hyaluronic acid, gellan gum, or cellulose.

The crosslinkable composition can include an amine functional polymer with a weight average molecular weight of greater than 1 kDa, greater than 5 kDa, greater than 10 kDa, greater than 25 kDa, greater than 50 kDa, greater than 100 kDa, or greater than 500 kDa. For example, the amine functional polymer may have a weight average molecular weight of from 1 to 500 kDa, or from 1 to 100 kDa, or from 2 to 50 kDa, or may have any weight average molecular weight encompassed by foregoing ranges.

In some embodiments, the crosslinkable composition includes a polymer in a weight % concentration of greater than 5 wt %, 10 wt %, 20 wt %, 30 wt %, 40 wt %, 50 wt %, 60 wt %, 70 wt %, 80 wt %, or 90 wt % based on the total weight of coating. In some embodiments, the crosslinkable composition includes the polymer in a weight % concentration ranging from 5-25 wt %, 10-30%, 20-40%, 1-50 wt %, 20-80 wt %, 45-90 wt %, 5-100%, or may have any concentration encompassed by the foregoing ranges. In some embodiments, the crosslinkable composition includes the polymer in a weight % concentration of about 20 wt %, 30 wt %, 40 wt %, 50 wt %, or 60 wt %.

In one illustrative example, the polymer can be an amine functional polymer. For example, the crosslinkable composition may include an amine functional polymer in a weight % concentration of greater than 5 wt %, 10 wt %, 20 wt %, 30 wt %, 40 wt %, 50 wt %, 60 wt %, 70 wt %, 80 wt %, or 90 wt % based on the total weight of coating. In some embodiments, the crosslinkable composition includes an amine functional polymer in a weight % concentration ranging from 5-25 wt %, 10-30%, 20-40%, 1-50 wt %, 20-80 wt %, 45-90 wt %, 5-100%, or may have any concentration encompassed by the foregoing ranges. In some embodiments, the crosslinkable composition includes an amine functional polymer in a weight % concentration of about 20 wt %, 30 wt %, 40 wt %, 50 wt %, or 60 wt %.

In some embodiments, the coating further includes a compound including a functionalized monomer, the functionalized monomer comprising an amine group, a carbonyl group, an aldehyde group, an ester group, an acrylate group, a hydroxyl group, a carboxyl group, a sulfonyl group, or a thiol group.

To functionalize the inner or outer surface of the implantable bioabsorbable material, the amine functional polymer may be applied to or coated on to the surface by a variety of known processes including spraying, dip-coating, powder coating, dispersion coating, lamination to other substrates, extrusion, molding, compression molding, or any other suitable means. It can be applied as very thin coatings, even when loaded with additive materials, and as such it enables implantable article, such as an implantable article including bioabsorbable material, to be made with minimal effect on the thickness and profile of the implantable bioabsorbable material. The coating may be applied in multiple layers, such as 1, 2, 3, 4, 5, 6, 7, 8, 9 or more 10 layers. The amount and composition applied which each layer may be the same or different as described herein.

As noted, the crosslinking composition may be capable of crosslinking to a protein supplied by a biological tissue. The crosslinkable composition can react with a crosslinkable moiety on the side chain of an amino acid, such as an amine group, a carboxylic acid group, a hydroxyl group, a thiol group, or any combination thereof. As an alternative example to tissue binding, blood may provide the biological crosslinkable moiety, in which case the implantable article or tissue adhesive system is a blood-activated implantable article or tissue adhesive system.

In some examples, the implantable article can be sealed or fixed onto a biological tissue via separate addition of a multi-branched crosslinker composition. That is, the crosslinking composition of the implantable article is configured to crosslink to an interface including biological proteins, such as blood or tissue in a human subject, and a multi-branched crosslinker composition may optionally be added. The multi-branched crosslinker can be used to tune the adhesive strength of the implantable article to the biological tissue. The multi-branched crosslinker composition may include a solvent, a buffer, or any pharmaceutically acceptable excipient.

As non-limiting examples, the multi-branched crosslinker composition may include a reactive PEG derivative, a reactive natural product or derivative, and any combinations thereof. Exemplary reactive PEG derivatives include but are not limited to poly(ethylene glycol) N-hydroxysuccinimide ester (PEG-NHS), multi-arm PEG-NHS, poly(ethylene glycol) aldehyde (PEG-CHO), multi-arm PEG-CHO, or PEG-maleimide.

The multi-arm PEG can be a three-arm, four-arm, six-arm, and eight-arm PEG molecule. The PEG derivative may have a molecular weight of up to 100 kDa, 75 kDa, 50 kDa, 25 kDa, 15 kDa, 10 kDa, or 5 kDa. For example, the PEG derivative may have a molecular weight ranging from 1-5 kDa, 1-10 kDa, 1-20 kDa, 1-30 kDa, 1-40 kDa, 1-50 kDa, 5-15 kDa, 10-25 kDa, or 10-20 kDa.

The multi-branched crosslinker composition can include a reactive natural product or derivative. Exemplary reactive natural products or derivatives include but are not limited to a quinone, a flavonoid, a coumarin, or a lignin. In representative examples, the reactive natural product or derivative includes genipin, tannic acid, or glyoxal.

The multi-branched crosslinker composition can include synthetic or natural polymers and copolymers. Exemplary components of a multi-branched crosslinker composition include amine-functionalized polymers and copolymers, including but not limited to Poly(N-isopropylacrylamide) amine terminated, Poly(ethylene glycol) bis(3-aminopropyl) terminated, hyaluronic acid, gellan gum, cellulose; copolymers containing amine-functionalized polymers like Polyvinylamine, those containing amine reactive functional groups, e.g., aldehyde, N-hydroxysuccinimide, isocyanate, and mixtures thereof.

The implantable article may be composed of a bioabsorbable material or a non-absorbable material. In some cases, the implantable article can include both bioabsorbable material and non-absorbable material. The bioabsorbable material and/or the non-absorbable may be biocompatible, antibacterial, anti-inflammatory, or conductive to the body's healing process.

Exemplary bioabsorbable materials can include polymers having lactic acid monomers. Hydrolytically degradable bioabsorbable materials include but are not limited to poly(lactic acid) (PLA), poly(glycolic acid) (PGA), poly(lactic-co-glycolic acid) (PLGA), polydioxanone (PDO), polycaprolactone (PCL), poly(α-hydroxy esters), trimethylene carbonate (TMC), poly(L-lactide-co-ϵ-caprolactone) (PLCL), poly(D,L-lactic acid) (PDLLA), poly(L-lactic acid) (PLLA), poly(D-lactic acid) (PDLA), poly(glycolide-co-trimethylene carbonate) (PGTMC), Polyethylene terephthalate (PET) and combinations thereof.

Exemplary enzymatically degradable bioabsorbable materials include but are not limited to polyhydroxyalkanoates (PHAs), hyaluronic acid, collagen, gelatin, cellulose, and combinations thereof.

Exemplary Reactive oxygen species (ROS) degradable bioabsorbable materials include but are not limited to polyethylene, polypropylene, poly(propylene sulfide), poly(thioether), polyether, poly(thioketal), poly(L-proline), poly(L-methionine) and combinations thereof.

Such bioabsorbable materials are widely used in various implantable medical devices, such as sutures, meshes, stents, orthopedic fixation devices, and drug delivery systems due to their ability to degrade safely within the body over a controlled period. Their degradation rates and mechanical profile can be tailored by adjusting polymer composition, molecular weight, and crystallinity, among other properties.

Exemplary non-absorbable materials can include polymers such as but not limited to polyurethane (PU), polyamide (PA), polytetrafluoroethylene (PTFE), polyetheretherketone (PEEK), polyaryletherketone (PAEK), polyetherimide (PEI), polyethersulfone (PES), polyetherketoneketone (PEKK), fluorinated ethylene propylene (FEP) and combinations thereof. In some examples, the non-absorabable materials can include an expanded polymer, e.g., expanded polytetrafluoroethylene (ePTFE), expanded polyethylene (ePE), expanded PEEK (ePEEK), and the like.

The implantable material may include a bioabsorbable material. A suitable bioabsorbable material can facilitate or promote tissue ingrowth after implantation of a device, for example. Components of the bioabsorbable material may include, but are not limited to: copolymers and homopolymers of poly (α-hydroxy esters), such as copolymers of poly(lactic-co-glycolic acid) (PLGA), poly(glycolic acid) (PGA), and poly(lactic acid) (PLA); trimethylene carbonate (TMC); copolymers of PLA and TMC (PLA: TMC), copolymers of PGA and TMC (PGA: TMC) and copolymers of PLGA and TMC; and combinations thereof.

In some embodiments, the bioabsorbable material may be synthesized through a sequential addition ring opening polymerization and composed of PGA and TMC, to form a highly porous, stretched, self-cohered, non-woven bioabsorbable web material; wherein A includes between 40 and 85 weight percent of the total weight, and wherein PGA includes glycolide recurring units; and TMC includes the remainder of the total weight and includes trimethylene carbonate recurring units said material being bioabsorbable and implantable.

Preferred bioabsorbable material materials are made with PGA: TMC copolymers having ratios of PGA to TMC of sixty-seven percent (67%) to thirty three percent (33%) (67:33-PGA: TMC) and fifty percent (50%) PGA to fifty percent (50%) TMC (50:50-PGA: TMC). The inherent viscosity of these polymers at 30° C. in hexafluoroisopropanol (HFIP), can range from an average of 0.5 dl/g to over 1.5 dl/g, and for preferred use can range from 1.0 dl/g to 1.2 dl/g. The acceptable melting point for this particular range of copolymer compositions as determined through a DSC melt peak can range from approximately 170° C. to 220° C. These copolymers' cumulative thermal exposure over time, be it from extrusion or other processing, needs to be minimized sufficiently to prevent transesterification reactions that can result in degradation of the copolymers' block structure and their correlating crystallinity and phase immiscibility characteristics.

The implantable article, specifically the bioabsorbable material or the crosslinkable composition, can further include chemically reactive groups that can serve as attachment sites for chemically bonding yet other chemical compositions, such as biologically active substances. These “bioactive substances” include enzymes, organic catalysts, ribozymes, organometallics, proteins, glycoproteins, peptides, polyamino acids, antibodies, nucleic acids, steroidal molecules, antibiotics, antimycotics, cytokines, carbohydrates, oleophobics, lipids, extracellular matrix material and/or its individual components, pharmaceuticals, and therapeutics. A preferred chemically-based bioactive substance is dexamethasone. Cells, such as, mammalian cells, reptilian cells, amphibian cells, avian cells, insect cells, planktonic cells, cells from non-mammalian marine vertebrates and invertebrates, plant cells, microbial cells, protists, genetically engineered cells, and organelles, such as mitochondria, are also bioactive substances. In addition, non-cellular biological entities, such as viruses, virions, and prions are considered bioactive substances.

Various other materials may be implemented that exhibit certain properties for facilitating at least some of the properties and functionalities of the bioabsorbable material after processing. At least some of the properties that facilitate the final functionality and properties of the bioabsorbable material are described herein in more detail. In certain illustrative examples, such bioactive substances can be encapsulated or entrapped within the matrices of an implantable article. In other examples, the bioactive substances can be immobilized on the surface of the implantable article. Enzymes and other bioactive substances can be covalently linked to the implantable article, such as to amine or carboxyl groups on materials like collagen, gelatin or chitosan scaffolds associated with the implantable article.

The implantable article 100 may be composed of a bioabsorbable material. The bioabsorbable material may include various properties suitable for the application in which it is applied. For example, the body 102 of the bioabsorbable material may be woven or non-woven. The body 102 of the bioabsorbable material may be formed as a clump or network of fibrils or filaments. The body 102 of the bioabsorbable material can be pleated and/or have a stored length. The amount of stored length is tunable during processing to facilitate the ability to provide for different amount of stretch or expansion during use. Because stored length may be facilitated by pleats and/or folds, it is generally understood that the thickness of the body 102 of the bioabsorbable material is variable depending on expansion or release of the pleats. However, during release of the stored length, the thickness or structure of the body 102 of the bioabsorbable material is generally consistent. For example, the body 102 of the bioabsorbable material may include a microstructure defined by the filaments, where the microstructure is substantially uncollapsed during expansion. This allows the bioabsorbable material to retain many of its properties and functionalities such as porosity, pore size, plushness, cellular ingrowth, and so forth.

In some examples, the filaments defining the body 102 may include melt-formed continuous filaments intermingled to form a porous web, wherein the melt-formed continuous filaments are self-cohered to each other at multiple contact points. Those filaments may be laid to form both the microstructure of the body 102 as well as any pleats and folds (e.g., via three-dimensional printing) or the microstructure may be formed and then the body 102 may be processed according to methods discussed herein to define the pleats and folds. In some examples, the melt-formed continuous filaments include at least one semi-crystalline polymeric component covalently bonded to or blended with at least one amorphous polymeric component. The melt-formed continuous filaments may possess partial to full polymeric component phase immiscibility when in a crystalline state. The body 102 can be processed or treated to instigate the crystalline state of at least some of the filaments. Otherwise stated, the orientation of the filaments can be set in the crystalline state to substantially retain the shape of any pleats and folds.

In some embodiments, the body 102 may be provided with various porosities. For example, in some embodiments, the body 102 may have a percent porosity greater than 70%, greater than 80%, greater than 90%, or any other suitable range therebetween. The porosity of the body 102 is defined within the three-dimensional microstructure. Because the body 102 includes a thickness that is greater than a single filament or even multiple stacked filaments, the pores are defined through a thickness of the body 102. In some embodiments, the body 102 may be provided with various thicknesses. For example, in some embodiments, the body 102 may be at least 0.10 mm thick, at least 0.20 mm thick, at least 0.30 mm thick, at least 0.40 mm thick, at least 0.50 mm thick, at least 0.60 mm thick, at least 0.70 mm thick, at least 0.80 mm thick, at least 0.90 mm thick, at least 1.00 mm thick, at least 2.00 mm thick, or at least 3.00 mm thick. The three-dimensional microstructure of the body 102 may include a network of pores throughout, including throughout the thickness of the body 102. Because of the three-dimensional character of the body 102 and the porous structure defined therein, the body 102 may provide various functionalities. For example, when used in surgical applications, the three-dimensional porous structure may facilitate tissue ingrowth and incorporation, which can provide for faster healing, increased adhesion, and so forth.

In addition to the body 102 including a three-dimensional structure through the thickness (e.g., Z-axis), in some embodiments the body 102 can be provided with a bulk, or overall three-dimensional shape or structure. For example, the body 102 may define a three-dimensional structure useful for the application in which it is to be used. The three-dimensional structure may include at least one of: a tubular construct, a sphere, a hemisphere, a partial sphere, a spheroid, a hemispheroid, a partial spheroid, an ellipsoid, a hemi-ellipsoid, a partial ellipsoid, a cone, and a partial dome. In other embodiments, the body 102 may be provided as a sheet having a substantially planar structure.

In some examples, the stored length of the body 102 is recoverable stored length. When the body 102 is expanded and released, the body 102 is capable of recovering at least some of the stored length. For example, the pleats and folds are at least partially recovered after expansion. In some embodiments, not all of the original stored length is recovered after the first expansion. In some embodiments, an article including stored length demonstrates substantially the same recovery of stored length following expansion and release cycle over a plurality of subsequent cycle. In other words, the article retains its recover properties, and the recovery property remains substantially the same, over a plurality of repetitions or cycles. In some embodiments, the stored length of the material is facilitated by the orientation of the filaments and specifically the orientation of the filaments being at least partially locked into the crystalline state of the material. Bioabsorbable filaments may be implemented in connection with the processes described therein and may result in substrates implemented in further processing as described herein.

In some embodiments, the implantable bioabsorbable material includes an outer surface 104 and a coating 106 on at least a portion of the outer surface 104. For example, the coating 106 may be applied to at least 10%, at least 20%, at least 30%, at least 40%, at least 50%, at least 60%, at least 70%, at least 80%, at least 90%, or at 100% of the surface, or from 10% to 100%, or may be applied at any percentage encompassed by the forgoing. In some embodiment it may be advantageous to apply the coating 106 in a pattern, for example, dots, stripes, zig-zags pattern and the like.

In some embodiments, the implantable bioabsorbable material includes a coating shield configured to delay or prevent for about 0.1 seconds to about 20 minutes premature reactivity with a bodily fluid comprising a protein. For example, premature reactivity may be delayed or prevented for about 0.1 seconds, about 0.5 seconds, about 1 second, about 10 seconds, about 20 seconds, about 30 seconds, about 40 seconds, about 50 seconds, about 1 min, about 2 mins, about 3 mins, about 4 mins, about 5 mins, about 6 mins, about 7 mins, about 8 mins, about 9 mins, about 10 mins, about 11 mins, about 12 mins, about 13 mins, about 14 mins, about 15 mins, about 16 mins, about 17 mins, about 18 mins, about 19 mins, or about 20 mins. In some embodiments, premature reactivity may be delayed or prevented for from about 0.1 seconds to about 5 mins, from about 1 second to about 13 mins, from about 3 mins to about 10 mins, from about 5 mins to about 8 mins, or from about 7 mins to about 13 mins, from about 1 second to about 20 mins, from about 5 mins to about 20 mins, or from about 15 mins to about 20 mins.

In some embodiments, the protein includes a blood protein, a protein in synovial fluid, or combinations thereof. For example, the blood protein may include albumin, globulins, transferrin, haptoglobin, fibrinogen, C-reactive protein, ceruloplasmin, lipoproteins, complement proteins, or combinations thereof.

In some embodiments, the coating shield includes a chemical shield, a mechanical shield, or a combination thereof.

The chemical shield of the implantable article includes a second composition including a negatively charged polymer functionalized with crosslinkable groups. In some embodiments, the negatively charged polymer includes carboxyl groups, hydroxyl groups, sulfonate groups, or a combination thereof. In certain embodiments, the crosslinkable groups include amine reactive functional groups including N-hydroxy succinimide groups, aldehyde groups, isocyanate groups, or a combination thereof.

The second composition of the chemical shield may be applied to the coating that is on a surface of the bioabsorbable or non-absorbable material. In some embodiments, the surface is an inner surface of the bioabsorbable or non-absorbable material. In other embodiments, the surface is an outer surface of the bioabsorbable or non-absorbable material. For example, the second composition may be applied using dip coating, spin coating, spray coating, bar or blade coating, brush coating, or a combination thereof.

The negatively charged polymer of the second composition can temporarily bind to proteins in a bodily fluid, thereby creating a steric barrier that limits the access of the proteins in the bodily fluid to the coating including a crosslinkable composition that is on the inner or outer surface of the implantable article. The negatively charged polymer acts as a barrier and delays or prevents a premature reaction between the crosslinkable composition in the coating and the proteins in a bodily fluid, which preserves the adhesive functionality of the implantable article. When light pressure is applied during deployment of the implantable article, the resulting steric hinderance between the negatively charged polymer and the proteins in a bodily fluid is overcome, allowing the crosslinkable groups to interact with the free amines on the proteins in a bodily fluid, thereby crosslinking them together.

The second composition including a negatively charged polymer functionalized with crosslinkable groups may have a molecular weight of from about 5,000 Da to about 30,000 Da, e.g., about 5,000 Da, about 5,500 Da, about 6,000 Da, about 7,000 Da, about 7,500 Da, about 8,000 Da, about 8,500 Da, about 9,000 Da, about 9,500 Da, about 10,000 Da, about 10,500 Da, about 11,000 Da, about 11,500 Da, about 12,000 Da, about 12,500 Da, about 13,000 Da, about 13,500 Da, about 14,000 Da, about 14,500 Da, about 15,000 Da, about 17,000 Da, about 19,000 Da, about 21,000 Da, about 23,000 Da, about 25,000 Da, about 27,000 Da, about 29,000 Da, or about 30,000 Da. For example, the molecular weight of the negatively charged polymer may be from about 5,000 Da to about 14,500 Da, from about 6,000 Da to about 13,000 Da, from about 7,000 Da to about 12,000 Da, or from about 8,500 Da to about 10,000 Da.

In some embodiments, the second composition including a negatively charged polymer functionalized with crosslinkable groups may have a molecular weight of from about 130,000 Da to about 150,000 Da, e.g., about 130,000 Da, about 133,000 Da, about 135,000 Da, about 137,000 Da, about 140,000 Da, about 143,000 Da, about 145,000 Da, about 147,000 Da, or about 150,000 Da. For example, the molecular weight of the negatively charged polymer may be from about 130,000 Da to about 147,000 Da, from about 135,000 Da to about 143,000 Da, or from about 137,000 Da to about 140,000 Da.

The negatively charged polymer of the second composition may be present in from about 1 wt % to about 20 wt % based on the total weight of the second composition and the crosslinkable group. For example, the negatively charged polymer may be present in about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %. For example, the negatively charged polymer may be present in about 1 wt % to about 15 wt %, from about 3 wt % to about 12 wt %, from about 5 wt % to about 10 wt %, or from about 6 wt % to about 8 wt %.

The implantable article of the present application includes a bioabsorbable or non-absorbable material, the bioabsorbable or non-absorbable material comprising an inner or outer surface and a coating on at least a portion of the inner or outer surface, wherein the coating includes a crosslinkable composition, the crosslinkable composition comprising an amine functional polymer immobilized to the inner or outer surface by crosslinking with a compound comprising an aldehyde group. In some embodiments, a chemical shield is applied to the coating including the crosslinkable composition on the inner or outer surface of the implantable article. In some embodiments, the coating is applied to the inner or outer surface of the implantable article and a mixture of the chemical shield and the crosslinkable composition is applied to the coating on the inner or outer surface of the implantable article.

The ratio of negatively charged polymer in the second composition and the crosslinkable composition of the coating may be about 1:50, about 1:20, about 1:10, about 1:5, about 3:10, or about 2:5.

In some embodiments, the implantable article includes hydrophobic molecules or hydrophobic polymers which are applied to the second composition on the inner or outer surface of the bioabsorbable or non-absorbable material. In some embodiments, the hydrophobic molecules or hydrophobic polymers are co-formulated with the second composition. The hydrophobic molecules or hydrophobic polymers may include polycaprolactone, silicone oil, vitamin E, or a combination thereof. The hydrophobic molecule or hydrophobic polymers, when used in combination with the second composition, may prevent or delay premature reactivity with a bodily fluid including a protein.

In some embodiments, the use of a hydrophobic molecule or hydrophobic polymer in combination with the second composition may prevent or delay premature reactivity for about 1 min to about 35 mins, e.g., about 1 min, about 2 mins, about 3 mins, about 4 mins, about 5 mins, about 6 mins, about 7 mins, about 8 mins, about 9 mins, about 10 mins, about 11 mins, about 12 mins, about 13 mins, about 14 mins, about 15 mins, about 17 mins, about 19 mins, about 21 mins, about 23 mins, about 25 mins, about 27 mins, about 29 mins, about 31 mins, about 33 mins, or about 35 mins. For example, premature reactivity may be delayed or prevented for about 1 min to about 30 mins, from about 3 mins to about 30 mins, from about 8 mins to about 27 mins, from about 10 mins to about 20 mins, from about 17 mins to about 29 mins, from about 19 mins, to about 25 mins, or from about 21 mins to about 23 mins.

The mechanical shield of the implantable article includes a knitted zipper cover, a delivery sleeve, or a delivery sheath cover.

In some embodiments, the knitted zipper cover, delivery sleeve, or delivery sheath cover includes expanded polytetrafluoroethylene (ePTFE), expanded polyethylene (ePE), polyether ether ketone (PEEK), ultra-high molecular weight polyethylene (UHMW PE), polysulfone (PSU), poly(methyl methacrylate) (PMMA), polypropylene (PP), polyethylene terephthalate (PET), polylactic acid (PLA), polycaprolactone (PCL), polyurethane (PU), silicone, nylon, polyvinylidene fluoride, perfluoroalkoxy alkane, fluorinated ethylene propylene, or combinations thereof.

In some embodiments, the knitted zipper cover has a knit density of from about 20 to about 70 stitches per inch, e.g., about 20 stitches per inch, about 25 stitches per inch, about 30 stitches per inch, about 35 stitches per inch, about 40 stitches per inch, about 45 stitches per inch, about 50 stitches per inch, about 55 stitches per inch, about 60 stitches per inch, about 65 stitches per inch, or about stitches per inch. For example, the knit density may be from about 20 stitches per inch to about 65 stitches per inch, from about 30 stitches per inch to about 55 stitches per inch, from about 40 stitches per inch to about 50 stitches per inch, or from about 45 stitches per inch to about 49 stitches per inch.

In some embodiments, the stitch density may vary along the length of the knitted zipper cover, such that certain portions are more densely knit, e.g., up to about 70 stitches per inch, while other sections are less densely knit, e.g., as low as about 20 stitches per inch, to achieve desired functional or aesthetic characteristics. In some embodiments, the knitted zipper cover may be constructed using an up-and-back approach, forming two layers of knitted material, essentially creating a double-layered knitted zipper cover, where the stitch density of each layer may differ or remain consistent, e.g., from about 40 stitches per inch to about 65 stitches per inch for the outer layer, and from about 20 stitches per inch to about 50 stitches per inch for the inner layer.

The mechanical shield provides a physical barrier to isolate the adhesive surface of the implantable article from exposure to a bodily fluid including a protein until precise activation at a desired tissue location. In some embodiments, the knitted zipper cover has hydrophobic properties, such that the knitted zipper cover repels bodily fluids including a protein and prevents premature interaction with the implantable article. In some embodiments, the implantable article is inserted into an impermeable delivery sleeve, which can be removed or peeled back prior to deployment. In some embodiments, the implantable article is delivered using an introducer sheath, wherein the introducer sheath is sealed off with a delivery sheath cover comprising a membrane, such that when deployment is triggered, the article perforates the membrane, allowing the adhesive to make contact with a target tissue.

In some embodiments, the chemical shield may be applied to the mechanical shield to form a unified protection system. For example, the implantable article may be shielded using a knitted zipper cover, a delivery sleeve, or a delivery sheath cover, and a chemical shield, as described herein, may be applied to the knitted zipper cover, delivery sleeve, or delivery sheath cover, thereby providing an integrated approach that enhances efficiency and simplifies device design by embedding the chemical shield within the mechanical shield.

Further described therein is a tissue adhesion system including an implantable article comprising an inner or outer surface comprising a crosslinkable composition; and a multi-branched crosslinker composition, wherein the polymeric crosslinking composition is adapted to crosslink the crosslinkable composition to a biological tissue, e.g., in the presence of a biological fluid, such as blood. A biological tissue may be found in a mammal or reptile. The mammal may be a human or an animal. For examples, biological tissue may include cells which consist of cells of the same form and function, such as surface tissue (skin), epithelial tissue, myocardial, connective or stromal tissue, muscles, nerves, blood and cartilage. The biological tissue may also be an organ, such as the liver, kidneys, lungs, heart. Alternatively, the biological tissue includes a blood vessel.

In the adhesive system, crosslinking of the crosslinkable composition to a reactive functional group supplied by a biological tissue or fluid adheres the implantable article to the biological tissue. The implantable article may include a bioabsorbable material, a non-absorbable material, or a combination thereof, as described and understood herein.

Another embodiment of this disclosed include a kit including the implantable bioabsorbable material; and a multi-branched crosslinker composition as described and understood herein.

In some embodiments, the implantable article or the tissue adhesive system, or any component thereof may further include pharmacologically active ingredients such as analgesics with or without anti-inflammatory effects, antiphlogistics, antimicrobial substances, antimycotics, antiparasitic substances. Active ingredients can be present as a pure active ingredient or in encapsulated form, for example in order to achieve a time-delayed release, depending on the application. Such active medicinal active substance may include a component which releases nitrogen monoxide under in vivo conditions, preferably L-arginine or an L-arginine-containing component or an L-arginine-releasing component, particularly preferably L-arginine hydrochloride. Proline, ornithine and/or other biogenic intermediates such as biogenic polyamines (spermine, spermite, putrescine or bioactive artificial polyamines) can also be used.

Other active ingredients may include vitamins or provitamins, carotenoids, analgesics, antiseptics, haemostatics, antihistamines, antimicrobial metals or their salts, plant substances or mixtures of substances that promote wound healing, plant extracts, enzymes, growth factors, enzyme inhibitors and combinations thereof. Analgesics include, in particular, non-steroidal analgesics, in particular salicylic acid, acetylsalicylic acid and their derivatives, e.g. Aspirin®, aniline and its derivatives, acetaminophen, e.g. Paracetamol®, antranilic acid and its derivatives, e.g. mefenamic acid, pyrazole or its derivatives, e.g. methamizol, Novalgin®, Phenazone, Antipyrin®, Isopropyl phenazone and very particularly preferably arylacetic acids and their derivatives, Heteroa rylessigsäuren and their derivatives, arylpropionic acids and their derivatives and Her-teroarylpropionic acids and their derivatives, for example Indomethacin®, Diclophenac®, Ibuprofen®, Naxoprophen®, Indomethacin®, Ketoprofen®, Piroxicam®.

Growth factors may include, for example, aFGF (Acidic Fibroplast Growth Factor), EGF (Epidermal) Growth Factor), PDGF (Platelet Derived Growth Factor), rhPDGF-BB (Becaplermin), PDECGF (Platelet Derived Endothelial Cell Growth Factor), bFGF (basic fibroblast growth factor), TGF a; (Transforming Growth Factor alpha), TGF β (Transforming Growth Factor beta), KGF (Keratinocyte Growth Factor), IGF1/IGF2 (Insulin-Like Growth Factor) or TNF (Tumor Necrosis Factor).

Vitamins or provitamins may include fat-soluble or water-soluble vitamins, vitamin A, group of retinoids, provitamin A, group of carotenoids, in particular special β-carotene, vitamin E, group of tocopherols, in particular a tocopherol, β-tocopherol, y-tocopherol, d-tocopherol and a-tocotrienol, β-tocotrienol, y-tocotrienol and d-tocotrienol, vitamin K, phylloquinone, in particular phytomenadione or vegetable vitamin K, vitamin C, L-ascorbic acid, vitamin B1, thiamine, vitamin B2, riboflavin, vita min G, vitamin B3, niacin, nicotinic acid and nicotinamide, vitamin B5, pantothenic acid, provitamin B5, panthenol or dexpanthenol, vitamin B6, vitamin B7, vitamin H, biotin, vitamin B9, folic acid and combinations thereof.

Similarly, antiseptic may be included have a gemicidal, bactericidal, bacteriostatic, fungicidal, virucidal, virustatic and/or generally microbiocidal effect. Substances that are particularly suitable are those selected from the group consisting of resorcinol, iodine, iodine-povidone, chlorhexidine, benzalkonium chloride, benzoic acid, benzoyl peroxide or cethylpyridinium chloride. In addition, antimicrobial metals, in particular, can also be used as antiseptics. In particular, silver, copper or zinc and their salts, oxides or complexes can be used in combination or alone as antimicrobial metals.

Further, herbal active ingredients that promote wound healing are, in particular, chamomile extracts, witch hazel extracts, for example Hamamelis virgina, calendula extract, aloe extract, for example aloe vera, aloe barbadensis, aloe feroxoder or aloe vulgaris, green tea extracts, seaweed-Extract, for example red algae or green algae extract, avocado extract, myrrh extract, for example Commophora molmol, bamboo extracts and combinations thereof. The content of the active ingredients is based primarily on the medically required dose and also on the compatibility with the other components of the implantable articles and materials contemplated herein.

The aforementioned agents and active ingredients can be encapsulated or entrapped within the matrices of an implantable article. In other examples, the agents and active ingredients can be immobilized on the surface of the implantable article. Otherwise, enzymes and other bioactive substances can be covalently linked to the implantable article.

### Methods of Application

In some embodiments, the implantable bioabsorbable material or implantable article may be adhered to a tissue during a surgical procedure by positioning the implantable bioabsorbable material or implantable article to an adjacent tissue in a subject and applying a multi-branched crosslinker composition to at least a portion of the coating on at least a portion of the inner or outer surface of the implantable bioabsorbable material or the implantable article including a crosslinkable moiety, thereby adhering the implantable bioabsorbable material to the adjacent tissue. The multi-branched crosslinker composition may include a reactive PEG derivative, a reactive natural product or derivative, or a combination thereof.

In an exemplary embodiment, the reactive PEG derivative includes poly(ethylene glycol) N-hydroxysuccinimide ester (PEG-NHS), multi-arm PEG-NHS, poly(ethylene glycol) aldehyde (PEG-CHO), multi-arm PEG-CHO, or PEG-maleimide. The multi-arm PEG can be a three-arm, four-arm, six-arm, and eight-arm PEG molecule. The PEG derivative may have a molecular weight ranging from 1-50,000 g/mol (1 k-50,000k), 1-100,000 g/mol (1 k-100,000k), or 1-150,000 g/mol (1 k-150,000 k). For example, the PEG derivative can have a molecular weight ranging from 1,000-5,000 g/mol, 2,500-7,500 g/mol, 5,000-10,000 g/mol, 10,000-20,000 g/mol, 12,500-25,000 g/mol, 25,000-50,000 g/mol, 45,000-75,000 g/mol, 50,000-100,000 g/mol, or 75,000-100,000 g/mol.

In yet other embodiments, the multi-branched crosslinker composition includes a reactive natural product or derivative including a quinone, a flavonoid, a coumarin, or a lignin. The reactive natural product or derivative may include, for example, genipin, tannic acid, or glyoxal.

The step of adhering the implantable bioabsorbable material to the adjacent tissue may result in sufficient adhesion within less than 5 min, 4 min, 3 min, 2 min, 1 min, 30 second, 15 seconds, 10 seconds, 5 seconds, or less than 1 second. This fixation time may be adjusted by varying the concentration and type of amine functional polymer immobilized to the inner or outer surface of the implantable bioabsorbable material as well as the type and amount polymeric crosslinking composition applied. For example, fixation time or time to fixation can be influenced by certain properties of the implantable article, including but not limited to thickness, porosity and different forms such as non-distended and distended webs of bioabsorbable substrates.

The step of adhering may the implantable bioabsorbable material to the adjacent tissue may result in sufficient adhesion once a certain pressure is applied. For example, an applied pressure of more than 1 PSI and less than 20 PSI may result in sufficient adhesion, i.e., sufficient fixation can result from pressure applied in an amount ranging from 1-20 PSI, 5-15 PSI, 10-12 PSI, 1-5 PSI, 1-10 PSI, 5-10 PSI, 10-15 PSI, or 15-20 PSI, inclusive.

The strength of adhesion between the implantable bioabsorbable material and the adjacent tissue may be least 0.3 mPA, 0.4 mPA, 0.5 mPA, 0.6 mPA, 0.7 mPA, 0.8 mPA, 1 mPA, or greater than 5 mPA. For example, the strength of adhesion may be from 0.3 mPA to 5 mPA, or from 0.3 mPA to 1 mPA or may have any value encompassed by the foregoing ranges.

The surgical procedures contemplated herein include, for example, implantation of suture-less hernia mesh, implantation of a fistula plug, filling a diabetic foot ulcer wound, repairing a thoracic aortic aneurysm with no landing, repair of av fistula, defect closure in aorta, sealing of air-leaking lung lobe, sealing of air-leaking trachea, adhesion of drug patch/sensor to an organ, anastomosis of small intestine, sealing of fluid-leaking stomach, sealing and landing devices in large blood vessels, septal defect closure, aortic dissection repair, or fixation of a tissue reinforcement graft.

It is understood that the bioabsorbable material biodegrades or absorbs in a patient's body. The degradation or absorption time is longer than the healing time for required from the surgical procedure, or any wound to be closed, for example, a period of 1 week, 2 weeks, 3 weeks, or 4 weeks or more. For example, the bioabsorbable material or implantable articles contemplated herein sufficiently adhere to tissue within the body a subject for more than 1, 5, 10, 10, 30, 40 or 50 days.

### EXAMPLES

### Test Methods

### Matrix Tensile Strength and Adhesive Strength

Tensile break load was measured using an INSTRON 5500R tensile test machine equipped with flat-faced grips and a 10 N load cell. The gauge length was 30 mm and the cross-head speed was 10 mm/min. For longitudinal MTS measurements, the larger dimension of the sample was oriented in the calendering direction, which was designated the “machine direction”. For the transverse MTS measurements, the larger dimension of the sample was oriented perpendicular to the calendering direction, which was designated the “transverse direction”.

The sample from the density measurement was used for tensile testing. The sample dimensions were 20 mm in length, 6.35 mm in width, and approximately 1 mm thick. The effective thickness is calculated from the mass, the area, and the density of the sample. Two to three samples were then tested individually on the tensile tester. The average of the 2-3 maximum load (i.e., the peak force) measurements was reported. The longitudinal and transverse MTS were calculated using the following equation:

MTS=Tensile stress at maximum load, wherein the density of PLA is taken to be 1.23 g/mL.

Matrix modulus is calculated using the following equation: Matrix Modulus=(small strain slope of load-displacement curve/cross-section area)*(density of “Polymer”)/density of the sample.

### Example 1

### Preparation of Immobilized Amine Functionality Based on Polyethylene Imine (PEI) Crosslinked with Glutaraldehyde on a Bioabsorbable Material

The following solutions were prepared and subsequently applied to a bioabsorbable material containing 67% polyglycolic acid (PGA): 33% trimethylene carbonate (TMC):
(1) Solutions S1, S2, and S3:
S1: PEI solution—21 gm Water and add 1.4 gm PEI (MW 2000 Da PEI, Catalog 408727, Sigma-Aldrich) and adjust pH 10.5 using 10M NaOH and add 12.41 gm IPA (100%)
S2: PEI solution—21 gm Water and add 1.4 gm PEI (MW 25000 Da PEI, Catalog 408727, Sigma-Aldrich) and adjust pH 10.5 using 10M NaOH and add 12.41 gm IPA (100%)
S3: PEI solution—21 gm Water and add 1.4 gm PEI (MW 2000 Da PEI, Catalog 408727, Sigma-Aldrich) and adjust pH 10.5 using 10M NaOH and add 12.41 gm IPA (100%)
(2) 37.5 gm DI water adjust pH 10.5 using 1M NaOH (2 gm NaOH in 50 mL DI water—1M NaOH)
(3) 37.5 gm DI Water and adjust pH 10.5 using 1M NaOH and add 0.150 ml of glutaraldehyde (50%)
(4) S1, S2, and S3:
S1 and S2: 36.38 gm DI water and add 2.26 gm PEI (MW 25000 Da PEI, Catalog 408727, Sigma-Aldrich) and adjust pH 10.5 using 10M NaOH
S3: 36.38 gm DI water and add 2.26 gm PEI (MW 50000-100000 Da PEI, Catalog 408727, Sigma-Aldrich) and adjust pH 10.5 using 10M NaOH
(5) 37.5 gm DI water 0.412 gm Sodium monophosphate, 0.184 gm boric acid, 1.1 6gm PEI (MW 25000 Da bPEI, Catalog 408727, Sigma-Aldrich) and adjust pH 8 using 10M NaOH

Subsequently, a 6 inch×3 inch sample of the bioabsorbable material was spray-coated in sequence with solutions prepared above followed by the specified wait times: Spray solution (1) and wait 10 minutes, spray solution (2) and wait 2 minutes, Spray solution (3) and wait 5 minutes, spray solution (4) and wait 10 minutes, and lastly, spray solution (5) and wait 10 minutes. The adhesive strength of the amine-functionalized, spray-coated bioabsorbable material was tested for adhesion with a biological tissue (porcine aorta).

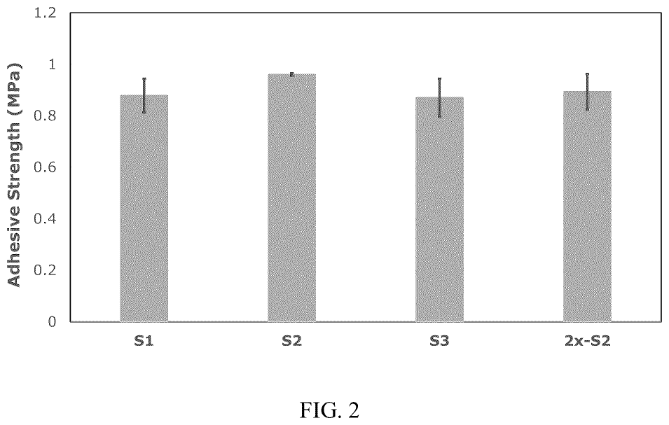

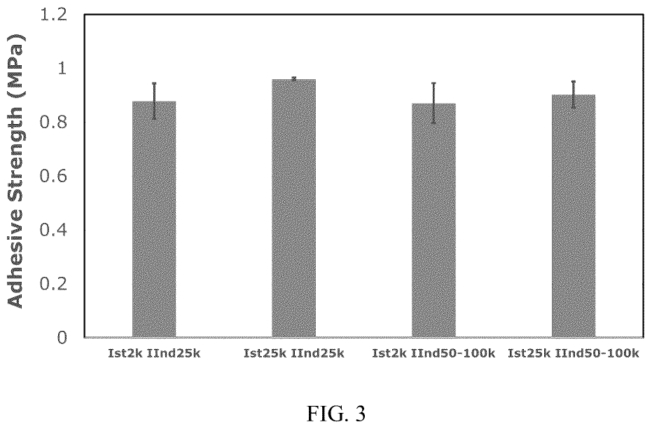

For testing the adhesive strength of the amine-functionalized bioabsorbable material to tissue, tissue and the amine-functionalized bioabsorbable were cut in the strip size of more than 20 mm (length)×6.35 mm (width). Separately, crosslinkers (PEG-NHS 4-arm 10 kDa) were dissolved in PBS at 30 wt % concentrations. Dissolved crosslinker was applied to tissue and amine-functionalized bioabsorbable on the area of 20 mm (length)×6.35 mm (width) and immediately adhered to each other by applying pressure by finger for 1 minute. Subsequently, adhered amine-functionalized bioabsorbable and tissue were tested for adhesive strength on Instron. Passed test was defined as complete detachment of both amine-functionalized bioabsorbable and tissue after testing at Instron, and failed test defined as top (imminent) layer of amine-functionalized bioabsorbable did not detach from tissue after testing at Instron, thus failed test will have adhesive strength higher relative to adhesive strength calculated from Instron testing. The results are shown in FIGS. 2 and 3.

### Example 2

### Adhesive Strength of Functionalized Materials

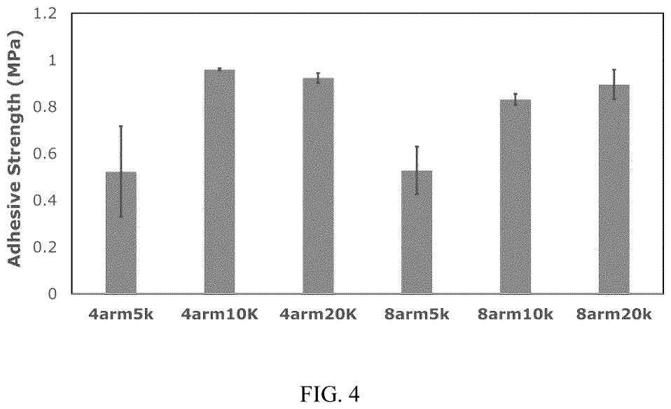

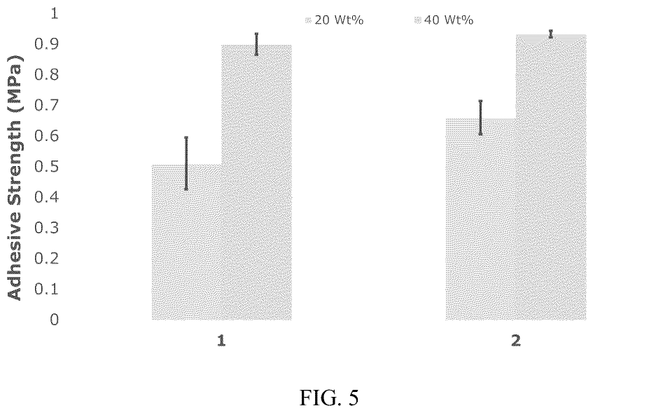

Amine functional groups containing branched polyethylene imine (PEI) was immobilized via chemical crosslinking with glutaraldehyde to the bioabsorbable material according to Example 1. The adhesive strength of the amine functionalized materials was tested for adhesion with a biological tissue (aorta) using the N-hydroxysuccinimide functionalized multi-arms (4 and 8 arms) polyethylene glycol (PEG) as an intermediate crosslinker, varying the PEG molecular weight, specifically between 5 k, 10 k and 20 k Da. The results are shown in FIG. 4. Further, it is demonstrated that the adhesive strength can be adjusted by varying the concentration of crosslinker applied, as shown in FIG. 5.

The adhesive strength of functionalized materials with tissue were tested on Instron according to the test method described above.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

### Example 3

### Preparation of Immobilized Amine Functionality Based on Polyethylene Imine (PEI) Crosslinked With Glutaraldehyde on a Bioabsorbable Material With a Chemical Shield

The PEI coated substrate was prepared following the procedures in Example 1.

7.656 g of Polyacrylic acid (PAA) (30 wt % MW 30000, Catalog 24771-250, Polysciences) was weighed out and added to 45 ml of DI water. The pH was adjusted around 4.5 using NaOH (10M). 1290 mg of 1-Ethyl-3-(3-dimethylaminopropyl) carbodiimide (EDC) ((Catalog-TS17144-0100, VWR)_was weighed out and dissolved in 5 ml DI water and added to the PAA solution drop-wise and stirred for 45 min (pH was 4.30). N-hydroxysuccinimide ((Catalog-AAA 10312-22, VWR) (1908 mg) was weighed out and dissolved in 5 ml DI water and added to the reaction mixture drop wise. The pH was adjusted to 4.0 using 10% (v/v) HCl. The mixture was stirred for 24 hr at room temperature, followed by dialysis in DI water for two days, and then freeze dried. Then, solutions of PAA-NHS (30000 Da) were prepared in 2.5, 5, 10, 15 wt % in saline (0.9 wt %): isopropanol (20:80 v/v) and then 50 wt % crosslinker (4arm 20 k PEG NHS) was added to the PAA-NHS solution. Finally, the PAA-NHS/crosslinker solution was painted onto the PEI coated substrates and dried for 1-2 hr at room temperature. Next, adhesive strength of PAA-NHS/crosslinker immobilized substrates as described in Example 1.

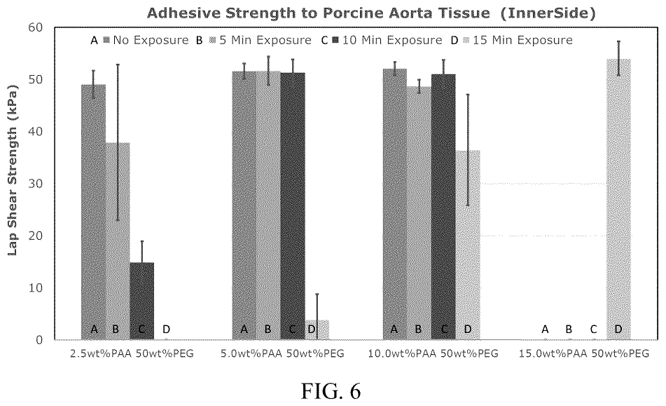

The results are shown in FIG. 6.

### Example 4

### Adhesive Strength of PEI coating on ePTFE, ePE, PET and NexED Bio A.

The following solutions were prepared and subsequently applied to the respective materials.

Coating of Polyethylene Imine on ePTFE and ePE
S1: PEI solution−21 gm Water and add 1.4 gm of PEI (Catalog number 06088 POLYETHYLENIMINE 100GRAMS, MW 1200 Da bPEI 1200,Polysciences)+21 gm iospropanol and adjust pH 10.5 using 10M NaOH and add 12.41 gm IPA (100%). (after coating wait for 10 minutes)
S2: 37.5 gm DI Water and adjust pH 10.5 using 1M NaOH and add 0. 150 ml of glutaraldehyde (50%)
S3: 36.38 gm DI water and add 2.26 gm PEI (Catalog number 06088 POLYETHYLENIMINE 100GRAMS, MW 1200 Da bPEI 1200, Polysciences) and adjust pH 10.5 using 10M NaOH.
S4: 37.5 gm DI water 0.412 gm Sodium monophosphate, 0.184 gm boric acid, 1.16 gm (Catalog number 06088 POLYETHYLENIMINE 100GRAMS, MW 1200Da bPEI 1200, Polysciences) and adjust pH 8 using 10M NaOH

Subsequently, a 6 inch×3 inch sample of the bioabsorbable material was spray-coated in sequence with solutions prepared above followed by the specified wait times: Spray solution (1) and wait 10 minutes, spray solution (2) and wait 2 minutes, Spray solution (3) and wait 5 minutes, spray solution (4) and wait 10 minutes, and lastly, spray solution (5) and wait 10 minutes. The adhesive strength of the amine-functionalized, spray-coated bioabsorbable material was tested for adhesion with a biological tissue (porcine aorta).

### Coating of Polyethylene Imine on PET and NexED BioA

S1: PEI solution-21 gm Water and add 1.4 gm PEI (MW 2000 Da PEI, Catalog 408727, Sigma-Aldrich) and adjust pH 10.5 using 10M NaOH and add 12.41 gm IPA (100%)
S2: 37.5 gm DI Water and adjust pH 10.5 using 1M NaOH and add 0.150 ml of glutaraldehyde (50%)
S3: 36.38 gm DI water and add 2.26 gm PEI (MW 25000 Da PEI, Catalog 408727, Sigma-Aldrich) and adjust pH 10.5 using 10M NaOH
S4: 37.5 gm DI water 0.412 gm Sodium monophosphate, 0.184 gm boric acid, 1.16 gm PEI (MW 25000 Da bPEI, Catalog 408727, Sigma-Aldrich) and adjust pH 8 using 10M NaOH

Subsequently, a 6 inch×3 inch substrate of each material was spray-coated in sequence with solutions prepared above followed by the specified wait times: Spray solution (1) and wait 10 minutes, spray solution (2) and wait 2 minutes, Spray solution (3) and wait 5 minutes, spray solution (4) and wait 10 minutes, and lastly, spray solution (5) and wait 10 minutes. The adhesive strength of the amine-functionalized, spray-coated bioabsorbable material was tested for adhesion with a biological tissue (porcine aorta).

### Adhesive (Lap Shear) Strength Of Bioadhesive Coated Substrates

To evaluate the adhesive strength of the bioadhesive-coated substrates to biological tissue, both the biological tissue (porcine aorta) and the functionalized Gore substrate were cut into rectangular strips of dimensions (Length: 20 mm; Width: 6.35 mm (¼ inch). The endothelial side of the porcine aorta was selected as the bonding interface, representing uniform and smooth moist tissue surface. Separately, crosslinkers (PEG-NHS 4-arm 10 kDa) were dissolved in PBS at 30 wt % concentrations. Dissolved crosslinker was applied to tissue and amine-functionalized bioabsorbable on the area of 20 mm (length)×6.35 mm (width) and immediately adhered to each other by applying pressure by finger for 1 minute.

Bioadhesive-coated substrates adhered to tissue were tested for adhesive strength using an INSTRON 5500R tensile test machine. Each condition was tested in triplicate. The tests were conducted with flat-faced grips and a 10 N load cell. The gauge length was set at 30 mm, and the crosshead speed was 10 mm/min. The tensile break load was recorded for each sample, and the average of the three maximum load (peak force) values was reported as the adhesive strength.

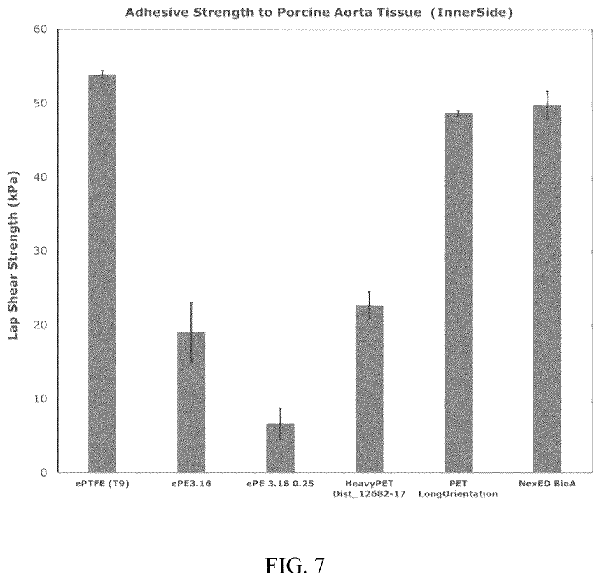

The results are shown in FIG. 7.

1. An implantable article comprising a bioabsorbable material, the bioabsorbable material comprising a surface and a coating on at least a portion of the surface,
wherein the coating comprises a crosslinkable composition, the crosslinkable composition comprising an amine functional polymer immobilized to the surface by crosslinking with a compound comprising an aldehyde group, wherein the surface can be an inner or outer surface.

2. The implantable article of claim 1, wherein the bioabsorbable material comprises poly (α-hydroxy esters), poly(lactic-co-glycolic acid) (PLGA), poly(glycolic acid) (PGA), poly(lactic acid) (PLA), trimethylene carbonate (TMC), or a combination thereof.

3. The implantable article of claim 1, wherein the amine functional polymer comprises polyethylenimine, poly(allylamine), poly(L-lysine), chitosan, poly(4-aminostyrene), poly(N-methylvinylamine), Oleylamine, lenoeylamine, dodecylamine, tris[(3 msalicylideneimino) ethyl]amine, polyvinylamine or a combination thereof; or
wherein the amine functional polymer comprises an amine modified polymer selected from a poly(ethylene glycol)-amine, Poly(N-isopropylacrylamide) amine terminated, poly(ethylene glycol) bis (3-aminopropyl) terminated, amine functionalized hyaluronic acid, gellan gum, or cellulose.

4. The implantable article of claim 1, wherein the crosslinkable composition further comprises a crosslinking intermediate, the crosslinking intermediate comprising an amine group, a succinimide group, a carbonyl group, an aldehyde group, an ester group, an acrylate group, a methacrylate group, a hydroxyl group, a carboxyl group, an isocyanate group, a sulfonyl group, or a thiol group.

5. The implantable article of claim 1 having a three-dimensional structure, the three-dimensional structure comprising a planar structure, an ellipsoid, a hemi-ellipsoid, a partial ellipsoid, a cone, a partial dome, a tubular construct, a sphere, a hemisphere, a partial sphere, a spheroid, a hemispheroid, or a partial spheroid.

6. The implantable article of claim 1, wherein the bioabsorbable material comprises a porosity of greater than 70%, greater than 80%, or greater than 90%.

7. The implantable article of claim 1,
wherein the bioabsorbable material comprises poly(lactic acid) (PLA), poly(glycolic acid) (PGA), poly(lactic-co-glycolic acid) (PLGA), polydioxanone (PDO), polycaprolactone (PCL), poly(α-hydroxy esters), trimethylene carbonate (TMC), poly(L-lactide-co-ϵ-caprolactone) (PLCL), poly(D,L-lactic acid) (PDLLA), poly(L-lactic acid) (PLLA), poly(D-lactic acid) (PDLA), poly(glycolide-co-trimethylene carbonate) (PGTMC), polyethylene terephthalate (PET), polyhydroxyalkanoates (PHAs), hyaluronic acid, collagen, gelatin, cellulose, polyethylene, polypropylene, poly(propylene sulfide), poly(thioether), polyether, poly(thioketal), poly (L-proline), poly (L-methionine), or a combination thereof, and
wherein the amine functional polymer comprises polyethylenimine covalently immobilized to the outer surface by crosslinking with glutaraldehyde.

8. An implantable article comprising a non-absorbable material, the implantable article comprising a surface and a coating on at least a portion of the surface,
wherein the coating comprises a crosslinkable composition, the crosslinkable composition comprising an amine functional polymer immobilized to the surface by crosslinking with a compound comprising an aldehyde group, and wherein the surface can be an inner or outer surface, optionally wherein the implantable article further comprises a coating shield configured to delay or prevent for about 1 to about 15 mins premature reactivity with a bodily fluid comprising a protein, and wherein the coating shield comprises a chemical shield, a mechanical shield, or a combination thereof.

9. The implantable article of claim 8, wherein the non-absorbable material comprises polyurethane (PU), polyamide (PA), polytetrafluoroethylene (PTFE), polyetheretherketone (PEEK), polyaryletherketone (PAEK), polyetherimide (PEI), polyethersulfone (PES), polyetherketoneketone (PEKK), an expanded polymer thereof, or a combination thereof.

10. The implantable article of claim 8, wherein the amine functional polymer comprises polyethylenimine, poly(allylamine), poly(L-lysine), chitosan, poly(4-aminostyrene), poly(N-methylvinylamine), Oleylamine, lenoeylamine, dodecylamine, tris[(3 msalicylideneimino) ethyl]amine, polyvinylamine or a combination thereof; or
wherein the amine functional polymer comprises an amine modified polymer selected from a poly(ethylene glycol)-amine, Poly(N-isopropylacrylamide) amine terminated, poly(ethylene glycol) bis(3-aminopropyl) terminated, amine functionalized hyaluronic acid, gellan gum, or cellulose.

11. The implantable article of claim 8, wherein the crosslinkable composition further comprises a crosslinking intermediate, the crosslinking intermediate comprising an amine group, a succinimide group, a carbonyl group, an aldehyde group, an ester group, an acrylate group, a hydroxyl group, a carboxyl group, a sulfonyl group, or a thiol group.

12. The implantable article of claim 8, comprising a three-dimensional structure, the three-dimensional structure comprising a planar structure, an ellipsoid, a hemi-ellipsoid, a partial ellipsoid, a cone, a partial dome, a tubular construct, a sphere, a hemisphere, a partial sphere, a spheroid, a hemispheroid, or a partial spheroid.

13. The implantable article of claim 1, further comprising a coating shield configured to delay or prevent for about 1 to about 15 mins premature reactivity with a bodily fluid comprising a protein, and wherein the coating shield comprises a chemical shield, a mechanical shield, or a combination thereof.

14. (canceled)

15. (canceled)

16. The implantable article of claim 13, wherein the chemical shield comprises a second composition comprising a negatively charged polymer functionalized with crosslinkable groups, and wherein the second composition is on at least a portion of the coating that is on the outer surface of the bioabsorbable material.

17. (canceled)

18. The implantable article of claim 16, wherein the negatively charged polymer comprises carboxyl groups, hydroxyl groups, sulfonate groups, or a combination thereof, and wherein the crosslinkable groups comprise amine reactive functional groups selected from N-hydroxy succinimide groups, aldehyde groups, isocyanate groups, and combinations thereof.

19. The implantable article of claim 13, further comprising hydrophobic molecules or hydrophobic polymers, wherein the hydrophobic molecules or hydrophobic polymers are applied to the second composition that is on the outer surface of the bioabsorbable material or co-formulated with the second composition.

20. (canceled)

21. The implantable article of claim 15, wherein the mechanical shield comprises a knitted zipper cover, a delivery sleeve, or a delivery sheath cover.

22. The implantable article of claim 21, wherein the knitted zipper cover comprises expanded polytetrafluoroethylene (ePTFE), expanded polyethylene (ePE), polyether ether ketone (PEEK), ultra-high molecular weight polyethylene (UHMW PE), polysulfone (PSU), poly(methyl methacrylate) (PMMA), polypropylene (PP), polyethylene terephthalate (PET), polylactic acid (PLA), polycaprolactone (PCL), polyurethane (PU), silicone, nylon, polyvinylidene fluoride, perfluoroalkoxy alkane, fluorinated ethylene propylene, or combinations thereof, and wherein the knit density is about 20 to about 70 stitches per inch.

23-46. (canceled)

47. A kit comprising:
the implantable article of claim 1; and
a multi-branched crosslinker composition.

48-52. (canceled)

53. A method for preparing an implantable article comprising
providing an implantable article, optionally comprising a bioabsorbable material or a non-absorbable material, having a surface, and
applying a coating on at least a portion of the surface,
wherein the coating comprises an amine functional polymer immobilized to the surface, the amine functional polymer immobilized to the surface by crosslinking with a compound comprising an aldehyde functional group, wherein the coating optionally comprises a coating shield configured to delay or prevent for about 1 to about 15 mins premature reactivity with a bodily fluid comprising a protein, and wherein the surface can be an inner or outer surface.
